(19) United States
(12) Patent Application Publication
(10) Pub. No.: US 2026/0158086 A1
(43) Pub. Date: Jun. 11, 2026

(54) WHOLE CELL, HEAT-KILLED POSTBIOTIC BLEND

(71) Applicant: Biohm Health Inc., Cleveland, OH (US)

(72) Inventors: Afif Ghannoum, Moreland Hills, OH (US); Samuel Schatz, New York, NY (US); Robert Fernald, Lakewood, OH (US)

(21) Appl. No.: 19/178,999

(22) Filed: Apr. 15, 2025

Related U.S. Application Data
(60) Provisional application No. 63/634,023, filed on Apr. 15, 2024.

(51) Int. Cl. A61K 35/747; A61K 35/00; A61K 35/745; A61K 36/064; A61K 38/47; A61P 37/04

(52) U.S. Cl. CPC A61K 35/747; A61K 35/745; A61K 36/064; A61K 38/47; A61P 37/04; A61K 2035/115

(57) ABSTRACT

A composition may postbiotics comprising two or more of the following components: Lactobacillus acidophilus, Lactobacillus rhamnosus, and Saccharomyces boulardii; wherein the postbiotics are dead, whole-cell postbiotics. A method of providing to a subject an immunomodulatory, immune regulatory and/or immune boosting effect through administering the composition is also provided. A method of making the postbiotic composition is also provided.

### CROSS-REFERENCE TO RELATED APPLICATIONS

This application claims the benefit of priority to U.S. provisional application No. 63/634,023, filed on Apr. 15, 2024. This prior application is incorporated herein by reference.

### FIELD

This disclosure relates generally to a method and composition of improving or balancing a microbiome of a mammal gut. More particularly, the disclosure relates to a method of improving immune system response and health.

### BACKGROUND

The gut microbiome and its role in disease states and conditions has been explored. Various conditions, diets, diseases, hormonal changes have the potential to cause disruption in the gut microbiome. Probiotics, which are beneficial microorganisms, have the potential to restore this microbial balance and alleviate symptoms. Probiotic supplements are already available in the market, with claims to improve overall health. Typically, these products contain Lactobacillus and/or Bifidobacterium strains, which both have demonstrated the ability to restore microbial balance in the gut and alleviate general digestive issues.

In addition, biofilms form in the gut and cause general health problems and are a hindrance to efforts to address microbiome imbalances. Biofilms are formed when unicellular microorganisms live together and form a community that is protected by an exopolysaccharide (EPS) matrix. This EPS matrix typically is a conglomeration of proteins, polysaccharides and extracellular DNA. Biofilm-associated microorganisms differ from their planktonic (freely suspended) counterparts. It is believed that biofilm-forming cells co-aggregate with each other to form coordinated groups attached to a biotic or abiotic surface, the cells are surrounded by a protective EPS matrix, communicate effectively through quorum sensing, and have low metabolic activity that limits the impact of conventional antimicrobials (both antifungal and anti-bacterial agents) acting against actively metabolizing cells or cells in stationary phase.

Microorganisms, including bacteria, fungi, and archaea form biofilms. For a given subject (e.g., a human) many microorganisms (termed microbiota) exist on or within various regions of said subject, e.g., the gut, the skin, the vagina, the respiratory tract, and the mouth. Gastrointestinal microbiota are important for vitamin and metabolites biosynthesis and digestion of complex macromolecules such as polysaccharides. Gastrointestinal microbiota, under normal conditions, aid in prevention of colonization of pathogenic microorganisms and in maintaining the integrity and function of the intestinal barrier, and supporting the immune system.

However, undesirable or unbalanced biofilms may be involved in a significant percentage of human microbial infections (Potera, C. (1999) SCIENCE 283:1837-8). It has been proposed that four criteria define biofilm etiology during an infection. These include whether the pathogenic bacteria or fungi are surface associated or adherent to a substratum; whether the bacteria are in clusters, encased in a matrix of bacterial, fungal or host constituents; whether the infection is localized; and whether the infection is resistant to antibiotic and anti-fungal therapies despite the antimicrobial sensitivity of the constituent planktonic organisms (Parsek, M. R. & Singh, P. K. (2003) ANN REV. MICROBIOL. 57:677-701; Ghannoum, M., et al. (2015) MICROBIAL BIOFILMS. Second Ed., Am. Soc. for Microbiology).

With respect to biofilms in the gut, it is now believed that bacteria and fungi can exist as biofilms on the colonic epithelium, within the mucus layer covering it, and on food particles in the lumen (MacFarlane, S. & MacFarlane, G. T. (2006) APPL. ENVIRON. MICROBIOL. 72:6204-11; Probert, H. M. & Gibson, G. R. (2002) CURR. ISSUES INTEST. MICROBIOL. 3:23-7). The biofilms in the GI tract populated at least with pathogenic bacteria form a dense filamentous film encased within an exopolysaccharide (EPS) matrix. Such biofilms are impenetrable to antimicrobials and host immune cells, and are difficult to remove or treat.

Postbiotics differ from probiotics in that they are non-viable, i.e., dead, microbial products. Postbiotics differ in their mechanism of action from probiotics; probiotics can directly colonize the gut, or cause an effect as they move through the GI tract and metabolize various substrates whereas postbiotics exert their effects mainly through the bioactive components present within the dead cells or metabolites. Postbiotics are used in the dietary supplement, food and ingredient industries as stand alone or in combination with other ingredients to enhance gut health and/or support the microbiome. The lack of viability of postbiotics can be general benefit in that they offer enhanced stability over traditional probiotics and can be used in different types of products and formulas and have longer shelf lives, whereas traditional probiotics are more limited in such capacity.

Under the category of postbiotics, there are additionally several formats or sub-categories, including dead whole cell microorganisms, fractured cell microorganisms, and microbial byproducts of microbial organisms.

### SUMMARY

Various forms of probiotics and postbiotics have existed on the market and have been studied for their alleged effects on the ability to modulate immune health. Probiotics, as living organisms, differ in activity and usability (as explained above) from postbiotics and postbiotics compounds are typically single organism origin. Disclosed herein is a novel blend of whole, dead cell postbiotic organisms for microbiome treatment and support. In an embodiment, the postbiotic organisms have been heat killed and “washed” to reduce clumping, thus maximizing the whole cell surface area. An unexpected and synergistic advantage of this combination of organisms is that they show a significantly increased immune-boosting effect than single organisms alone. The combinations of these organisms impart a unique and novel effect on both RNA expression and cytokine production as seen in laboratory assays described in the Examples.

The inventors identified a novel combination of heat-killed whole cell organisms, Lactobacillus acidophilus. Lactobacillus rhamnosus, and/or Saccharomyces boulardii that impart an immunomodulatory, immune regulatory and/or immune boosting effect when used in combinations as shown in the Examples.

In some aspects, the techniques described herein relate to a composition including: postbiotics including two or more of the following components: Lactobacillus acidophilus, Lactobacillus rhamnosus, and Saccharomyces boulardii; wherein the postbiotics are dead, whole-cell postbiotics.

In some aspects, the techniques described herein relate to a method of providing to a subject an immunomodulatory, immune regulatory and/or immune boosting effect, the method including consumption by, or administration to, the subject a composition including postbiotics including two or more of the following components: Lactobacillus acidophilus, Lactobacillus rhamnosus, and Saccharomyces boulardii; wherein the postbiotics are dead whole-cell postbiotics.

In some aspects, the techniques described herein relate to a method of making a postbiotic product, the steps including: fermenting and cultivating live probiotic cells, the cells selected from two or more of the following components: Lactobacillus acidophilus, Lactobacillus rhamnosus, and Saccharomyces boulardii; heat treating the live probiotic cells suspended in solution to induce cell inactivation while maintaining integrity of the cells, thereby resulting in a whole-cell postbiotic product; and separating the whole-cell postbiotic product from the solution.

In another aspect, the techniques described herein related to a method of improving a gut barrier, the method comprising consumption by, or administration to, a subject a composition comprising postbiotics comprising two or more of the following components: Lactobacillus rhamnosus, and Saccharomyces boulardii; wherein the postbiotics are dead whole-cell postbiotics. In an embodiment, the improving of the gut barrier comprises reducing TNFa gene expression by the composition as compared to stimulation with lipopolysaccharide from E. coli.

Unless the context dictates otherwise, the terms composition, formulation, and dosage form can be used interchangeably herein. For example, it is contemplated that the composition or formulation consumed by, or administered to, a subject can be consumed or administered as a dosage form, for example, as a unit (for example, a spoonful) of the composition. Similarly, it is understood that a dosage form can, for example, comprise a composition or formulation described herein. For example, a dosage form can comprise, for example, a composition/formulation in the form of a powder described herein, or, for example, a capsule that contains such a composition/formulation.

Other features and advantages of the technology disclosed herein will be apparent from the following detailed description and claims.

### BRIEF DESCRIPTION OF THE DRAWINGS

FIG. 1 is a schematic representation of an exemplary process for making a postbiotic composition described herein.

FIG. 2 is a graph of control examples and unwashed, washed, and washed-in-house examples for fold change in TNF and IL6 gene expression.

FIG. 3 is a graph of another set of control examples and unwashed, washed, and washed-in-house examples for fold change in TNF and IL6 gene expression.

FIG. 4 is a graph showing other combinations of postbiotic components and the fold change in various gene expressions.

FIG. 5 shows data for postbiotic compositions comprising Lactobacillus acidophilus (LA) and Lactobacillus rhamnosus (LR) in 1:1 and 2:1 ratios and compares these to an LA:LR:Saccharomyces boulardii (SB) 1:1:1 composition.

FIG. 6 shows data for postbiotic compositions comprising LA:LR:SB in a 1:1:1 ratios and compares these to an LA, LR, and SB separately.

FIG. 7 shows data for postbiotic compositions comprising LA and LR in 1:1 ratios and compares these to an LA, LR, and SB separately.

### DETAILED DESCRIPTION

This disclosure pertains to a novel formulation including a combination of whole, dead cell, postbiotic organisms postbiotic strains. Optionally, the postbiotics can be combined with probiotics, dietary supplements, and food ingredients. Disclosed herein is a novel blend of whole, dead-cell, postbiotic organisms for microbiome treatment and support. In an embodiment, the postbiotic organisms have been heat killed and “washed” to reduce clumping, thus maximizing the whole cell surface area. An unexpected and synergistic advantage of this combination of organisms is that they show a significantly increased immune boosting effect than single organisms alone. The combinations of these organisms impart a unique and novel effect on both RNA expression and cytokine production as seen in laboratory assays described in the Examples.

A synergistic combination of heat-killed, whole-cell organisms, Lactobacillus acidophilus, Lactobacillus rhamnosus, and Saccharomyces boulardii that impart an immunomodulatory, immune regulatory and/or immune boosting effect when used in combinations are shown in the Examples. To test how each postbiotic strain and combination affects systemic inflammatory responses, samples of human peripheral blood mononuclear cells (PBMCs) were exposed to different concentrations of each postbiotic strain and then the expression of relative cytokines was measured via RNA-seq. Combinations of Lactobacillus acidophilus, Lactobacillus rhamnosus, and/or Saccharomyces boulardii were identified as providing a surprisingly greater benefit than the individual heat-killed cells alone.

Additionally, a Luminex 45-plex cytokine, chemokine, and growth factor panel was analyzed after incubating postbiotic combinations. The Luminex test results showed significantly improved activity in multiple cytokine and chemokine expression in the combinations of organisms versus the single organisms alone.

In an embodiment, the postbiotic product confers an immunological health benefit on the target host. Specifically the postbiotic product induces an immune response observed in vitro in primary human monocytic cells (“PBMC”). The postbiotic product is intended to be used as a dietary supplement or functional food or drink to support immune health by positively influencing their microbiome composition.

While the discussion and focus of this application is on improving immunity/immune system response in humans, the target subject may, more broadly, be a mammal (e.g., human, a companion animal (e.g., dog, cat, or rabbit), or a livestock animal (for example, cow, sheep, pig, goat, horse, donkey, and mule, buffalo, oxen, or camel)). In certain embodiments, the subject is a healthy subject. In an embodiment, the formulation comprises two or more postbiotics selected from: Lactobacillus acidophilus, Lactobacillus rhamnosus, and Saccharomyces boulardii; wherein the postbiotics are dead, whole-cell postbiotics. In an embodiment, all three of Lactobacillus acidophilus, Lactobacillus rhamnosus, and Saccharomyces boulardii are included in the formulation. In an embodiment, the postbiotics are originally live and heat-killed as described herein to preserve the whole cell. Described another way, the postbiotics are not killed by lysing, nor do they contain only the lysed cell contents. In an embodiment, the formulation also comprises a prebiotic fiber.

L. rhamnosus can be included in the composition in an amount of 0.1 to 10 billion CFUs (10 to 500 mg), or 0.01 to 15 billion CFUs, such as, 0.5 to 5 billion CFUS, or 1.5 to 3 billion CFUs. L. rhamnosus is a lactic acid non-pathogenic bacterium that is resistant to acid and bile and has growth characteristics that allow it to survive and persist within the human gastrointestinal tract. It also displays excellent adhesion to the intestinal epithelial layer to inhibit the growth and adherence of several known pathogens and has been reported to form biofilms.

L. acidophilous, e.g., can be included in the composition in an amount of 0.1 to 10 billion CFUs (10 to 500 mg), or 0.01 to 15 billion CFUs, such as, 0.5 to 5 billion CFUs, or 1.5 to 3 billion CFUs. L. acidophilous is non-pathogenic, Gram positive bacteria that is common in the human GI tract and oral cavity. Structurally, L. acidophilous is an immobile rod-shaped organism that ranges in size from 2-10 m in size. It has a phospholipid bilayer membrane with a cell wall comprising peptidoglycan. Teichoic acids and surface proteins are present in the cell wall along with anionic and neutral polysaccharides.

S. boulardii, e.g., can be included in the composition in an amount of 0.1 to 10 billion CFUs (10 to 500 mg), or 0.01 to 15 billion CFUs, such as, 0.5 to 5 billion CFUs, or 1.5 to 3 billion CFUs. S. boulardii is a tropical yeast, a non-pathogenic, non-spore-forming fungus in the division Ascomycota.

In an embodiment, a prebiotic fiber is also included in the postbiotic composition. Prebiotics are compounds that promote growth of beneficial organisms such as bacteria and fungi by acting as substrates for growth. Fiber that is non-digestible or poorly digestible in the upper gastrointestinal tract is often a prebiotic. A prebiotic is also a substance that is fermentable by substances on or in the body.

For the whole-cell, non-viable postbiotic composition disclosed herein, various ingredient combinations are recited below:
L. acidophilus+L. rhamnosus+S. boulardii
L. acidophilus+L. rhamnosus
L. rhamnosus+S. boulardii
L. acidophilus+S. boulardii
Prebiotic Fiber+L. acidophilus+L. rhamnosus+S. boulardii
Prebiotic Fiber+L. acidophilus+L. rhamnosus
Prebiotic Fiber+L. rhamnosus+S. boulardii
Prebiotic Fiber+L. acidophilus+S. boulardii
L. acidophilus+L. rhamnosus+S. boulardii
L. acidophilus+L. rhamnosus
L. rhamnosus+S. boulardii
L. acidophilus+S. boulardii
Prebiotic Fiber+L. acidophilus+L. rhamnosus+S. boulardii
Prebiotic Fiber+L. acidophilus+L. rhamnosus
Prebiotic Fiber+L. rhamnosus+S. boulardii
Prebiotic Fiber+L. acidophilus+S. boulardii

The various combinations of whole-cell, non-viable bacteria and/or fungi can each be in a dosage form in a total amount of 5 to 1000 mg, such as, for example, 10 to 300 mg, or 20 to 250 mg, 25 to 100 mg, or 150 mg to 250 mg. The various combinations of whole-cell, non-viable bacteria and/or fungi can each be in a dosage form in a total amount of 10 B to 800 B cells per gram, such as, for example, 25 B to 250 B, 50 B to 200 B, 210 B to 300 B, or 15 B to 50 B. In an embodiment, whole cell, non-viable S. cerevisiae is included as a substitute or in addition to S. boulardii.

In an embodiment, the prebiotic fiber may be present in an amount of 10 mg to 20,000 mg, such as 15 mg to 15,000 mg, 25 mg to 10,000, 50 mg to 1,000 mg, or 5,000 to 18,000 mg.

The prebiotic fiber may be selected from one or more of: arabinoxylan, beta-glucan, carboxymethylcellulose, cellulose, chicory root fiber, cottonseed fiber, edible bean powder, fructo-oligosaccharides, galacto-oligosaccharides, hydroxypropyl-methylcellulose, inulin (artichoke), inulin (chicory), methylcellulose, modified resistant starch, oligofructose, pea fiber, pectin, polydextrose, polyfructans, Psyllium, resistant starch, resistant dextrin, resistant maltodextrin, rice bran, short chain fructooligosaccharides, soluble corn fiber, soluble dextrin, soy fiber, wheat bran, xanthan gum, golden kiwi fiber, xylo-oligosaccharides, galactomannan (guar gum) and tapioca starch.

In certain embodiments, the composition comprises a whole cell, non-viable postbiotics selected from at least two of non-pathogenic fungal strain Saccharomyces boulardii 16 mxg, non-pathogenic bacterial strains of Lactobacillus rhamnosus 18 fx, and/or Lactobacillus acidophilus 16 axg.

In an embodiment, the whole-cell, non-viable postbiotic composition may be administered in a combination with 10 to 1,000 mg, e.g., 15 to 800 mg, or 21 to 630 mg, of a probiotic composition comprising Lactobacillus acidophilus 16 axg, Saccharomyces boulardii 16 mxg, Lactobacillus rhamnosus 18 fx, Bifidobacterium breve 19 Bx and amylase. This probiotic compound can be used in amount of 0.1 to 10 billion CFUs or 10-660 mg, such as, for example, or 0.01 to 15 billion CFUs, such as, 0.5 to 5 billion CFUs, or 1.5 to 3 billion CFUs. In an embodiment, the probiotic product is a commercial product known as BIOHM FX. In an embodiment, the particular strains used in BIOHM FX, e.g., Lactobacillus acidophilus, Lactobacillus rhamnosus, and Saccaromyces boulardii, are used in the present postbiotic composition, but as dead whole cell postbiotics and for immune system support.

The compositions disclosed herein or the optional viable BIOHM FX component or sub-components of the BIOHM FX component may be included to aid in remediation of biofilms in the gut. Biofilms may exist in the GI tract and can block nutrient absorption and effectiveness of probiotics. These biofilms can hinder efforts to remediate the decreased gut microorganisms found herein. In certain embodiments, compositions described herein can be used to disrupt a biofilm comprising, for example, pathogenic bacteria and fungi within the gastrointestinal (GI) tract (for example, esophagus, stomach, upper intestine, and/or the lower intestine) of a subject. In certain embodiments, the preselected region comprises the duodenum, the jejunum, and/or the ileum of the upper GI tract, whereas in other embodiments, the preselected region comprises the appendix, the proximal colon, and/or the rectum of the lower GI tract.

In an embodiment, the composition can also include non-pathogenic bacteria and fungi that improve nutrient (for example, protein, vitamin, mineral, and co-factor) absorption by mammalian (for example, human) intestinal epithelial cells. Some of these are listed below.

Additional probiotic additives in contemplated embodiments include those selected from the following: Lactobacillus casei, Lactobacillus plantarum, Lactobacillus acidophilus, Bacillus subtillis, Lactobacillus rhamnosus, Lactobacillus helveticus, Bifidobacterium longum, Bifidobacterium lactis, Bifidobacterium infantis, Bifidobacterium bifidum, Bifidobacterium adolescentis, Bifidobacterium breve, Saccaromyces boulardii, Brevebacillus laterosporus, and combinations thereof. All such probiotic organisms could be included in the formulation at levels of 0.1-10 billion CFU, and/or 10-500 mg.

In an embodiment, an enzyme may also be included in the composition. Certain enzymes disrupt extracellular matrix polysaccharides (EPSs) present in the biofilm making the pathogenic organisms more open to challenge by the non-pathogenic bacterial and fungal strains in the dosage forms. In certain embodiments, upon administration of the composition, the non-pathogenic bacterial strains (i) displace the pathogenic bacteria in the biofilm, (ii) interfere with the attachment of the pathogenic bacteria or fungi to a substratum of the biofilm, (iii) displace the pathogenic bacteria/fungi from an extracellular polymeric matrix present in the biofilm, (iv) prevent filamentation of the pathogenic fungi in the biofilm, or (v) a combination of any of the foregoing. In certain embodiments, administration of the composition permits restoration of the natural microbiome of the region in the subject and reduces the growth and/or development of pathogenic bacteria and/or pathogenic fungi.

The inclusion of such an enzyme is particularly useful if subject has a biofilm of pathogenic bacteria and/or fungi. The microorganisms within the biofilm that are disrupted by the composition may comprise any type of pathogenic microorganism, e.g., bacteria (for example, Gram-positive bacteria and Gram-negative bacteria), fungi (for example, yeast and mold), archaea, and protozoa.

In certain embodiments, the probiotic composition further comprises an enzyme capable of disrupting a biofilm in a subject. In certain embodiments, the enzyme is selected from the group consisting of amylase, cellulase, hemicellulase, lysozyme, pectinase, DNase I, Serratia peptidase or Serratiopeptidase, hemicellulase/pectinase complex, β-1,3-glucanase, acid protease, alkaline protease, glucoamylase, endoglucanase, xylanase, lipase, lysozyme, protease/peptidase complex, dipeptidyl peptidase IV (DPP-IV), chitosanase, bromelain, papain, kiWi protease actinidi, a plant-derived protease, zymolase, nuclease, and phytase. In certain embodiments, the enzyme is an amylase selected from the group consisting of Bacillus stearothermophilus amylase, Bacillus amyloliquefaciens amylase, Bacillus subtilis amylase, Bacillus licheniformi amylase, Aspergillus niger amylase, and Aspergillus oryzae amylase.

In an embodiment, one or more enzymes, such as bromelain, cellulase(es), lipase(es), papain, protease(es) amylase(es), galactosidase may be included in the 10-250 mg range,

In certain embodiments, the composition comprises about 500 SKB units of amylase, selected from α-Amylase, an endo-hydrolase that catalyzes the hydrolysis of internal α-1, 4-glycosidic linkages in starch to yield products like glucose and maltose, β-Amylase, an exo-hydrolase enzyme that hydrolyses α-1, 4-glucan linkages to yield successive maltose units, and T-Amylase, which cleaves α-1, 6-glycosidic linkages, in addition to cleaving the last α-1, 4-glycosidic linkages to yield glucose, or a combination thereof.

In certain embodiments, the probiotic composition comprises 100 to 5,000 SKB units of amylase, 200 to 4,000 SKB units of amylase, from 300 to 2,000 SKB units of amylase, or 400 to 1,000 SKB units of amylase. In certain embodiments, the composition comprises 500 SKB units of amylase. An SKB or Sandstedt, Kneen, and Blish unit refers to the amount of amylase to catalyze 1 μmole substrate per minute.

In certain embodiments, the probiotic composition comprises a cellulose, for example, and comprises 100 to 300 CU (Cellulase unit) units per unit composition, for example, 200 CU. In certain embodiments, composition comprises a hemicellulose/pectinase complex, and comprises 60 to 100 HSU (Hemicellulose Specific Units) units per unit composition, for example, 80 HSU. In certain embodiments, the composition comprises a 0-gluconase, and comprises 6 to 10 BGU (Beta Glucanase Unit), units per unit composition, for example, about 8 BGU. In certain embodiments, the composition comprises an acid protease, and comprises 15 to 25 SAP (Shrimp Alkaline Phosphatase) units per unit composition, for example, about 20 SAP units. In certain embodiments, the composition comprises alkaline protease, and comprises 15 to 25 HUT (hemoglobin unit Tyrosine base) units per unit composition, for example, about 20 HUT units.

The enzyme may be chosen depending upon the type of biofilm and the microorganisms disposed therein. For example, an amylase enzyme may be used to degrade or otherwise disrupt carbohydrate components of the biofilm, and a nuclease such a DNase I may be used for digesting or otherwise disrupt DNA in the biofilm.

In certain embodiments, the composition comprises two or more (e.g., 2, 3, 4, 5, or more) different enzymes selected from amylase, cellulase, hemicellulase, lysozyme, pectinase, DNase I, Serratia peptidase, Serratiopeptidase, hemicellulase/pectinase complex, β-1,3-glucanase, acid protease, alkaline protease, glucoamylase, endoglucanase, xylanase, lipase, lysozyme, protease/peptidase complex, dipeptidyl peptidase IV (DPP-IV), chitosanase, bromelain, papain, kiWi protease actinidi, a plant-derived protease, phytase, zymolase and nuclease.

In certain embodiments, total amount of cellulase administered, for example, by the administration of one or more units of the composition, may be 1 to 10,000 CU, the total amount of hemicellulase/pectinase complex administered, for example, by the administration of one or more units of the composition, may be 1 to 8,000 HSU, the total amount of 0-gluconase, for example, by the administration of one or more dosage units, may be 1 to 1000 BGU, the total amount of acid protease, for example, by the administration of one or more dosage units, may range be 1 to 10,000 SAP, and the total amount of alkaline protease, for example, by the administration of one or more dosage units, may be 1 to 40,000 HUT.

In certain embodiments, a component of the composition described herein is used to disrupt a biofilm disposed within the GI tract of a subject with an elevated relative abundance of pathogenic bacterial species such as Solobacteria moorei, E. coli, Serratia marcescens, and Ruminococcus gnavus in the biofilm disposed within the GI tract of the subject when compared to levels typically found in healthy subjects. For example, the GI tract biofilm may comprise: Candida tropicalis and Escherichia coli; Candida tropicalis and Serratia marcescens; Candida tropicalis, Escherichia coli, and Serratia marcescens; other Candida yeasts, Klebsiella oxytoca, or Solobacteria moorei.

In certain embodiments, a composition described herein comprises non-pathogenic bacterial strains for disrupting or replacing a biofilm containing two pathogenic bacterial species. For example, the biofilm targeted for disruption or replacement may contain E. coli and a Bacteroides spp. (e.g., Bacteroides coprophilus, Bacteroides eggerthii, Bacteroides ovatus, Bacteroides fragilis, Bacteroides plebeius, or Bacteroides uniformis), or Solobacteria moorei.

In certain embodiments, the abundance of pathogenic fungus Candida tropicalis in a biofilm disposed within the GI tract of a subject is elevated when compared to levels typically found in healthy subjects. In certain embodiments, biofilms within the GI tract containing C. tropicalis or C. Albicans combined with E. coli and/or S. marcescens are enriched in fungal hyphae, a form of growth associated with pathogenic conditions. Fungal filamentation can sometimes be a virulence factor used by Candida to damage host tissues and to trigger a specific host immune response. In certain embodiments, the dosage form of the invention is used to disrupt a biofilm disposed within the GI tract of a subject with an elevated relative abundance of bacterial species such as species of the Firmicutes phylum. In certain embodiments, the dosage form of the invention is used to disrupt a biofilm disposed within the GI tract of a subject with an elevated relative abundance of fungal species such as species of the Ascomycota phylum and species of the Zygomycota phylum and species of the Basidiomycota phylum.

The bacteria within the biofilm exist in intimate contact with the fungus but may differ in their specific interactions with the fungus. In certain embodiments, the pathogenic bacteria (e.g., E. coli) may be fused to the fungal cells within the biofilm. Alternatively, or in addition, the pathogenic bacteria and pathogenic fungi disposed within the biofilm may form a “digestive plaque,” where the bacteria and fungi are protected from antimicrobial drugs and the host's immune system. It is understood that digestive plaque can disrupt the normal or healthy microbiome of the GI tract, and cause or be otherwise associated with a GI disease or disorder.

In certain embodiments, one or more additional bacterial strains either in a probiotic or postbiotic form can be included in the postbiotic composition. These may comprise bacterial strains of any one or more of the following bacterial species: Agrococcus jenensis, Alistipes indistinctus, Alistipes massiliensis, Alkalibacterium iburiense, Anoxybacillus kestanbolensis, Bacillus cereus, Bacillus clausii, Bacillus Coagulans, Bacteroides coprophilus, Bacteroides eggerthii, Bacteroides ovatus, Bacteroides fragilis, Bacteroides plebeius, Bacteroides uniformis, Bifidobacterium adolescentis, Bifidobacterium animalis, Bifidobacterium breve, Bifidobacterium longum, Bifidobacterium pseudolongum, Blautia obeum, Blautia product, Candidatus azobacteroides, Candidatus portiere, Candidatus portiera, Clostridium celatum, Clostridium hiranonis, Clostridium neonatale, Clostridium perfringens, Clostridium tyrobutyricum, Collinsella aerofaciens, Collinsella stercoris, Coprococcus eutactus, Corynebacterium stationis, Desulfosporosinus meridiei, Desulfovibrio D168, Dorea formicigenerans, Eggerthella lenta, Erwinia oleae, Faecalibacterium prausnitzii, Lactobacillus agilis, Lactobacillus reuteri, Lactobacillus ruminis, Lactobacillus salivarius, Lactobacillus zeae, Listeria weihenstephanensis, Paenibacillus mucilaginosus, Parabacteroides distasonis, Pediococcus acidilactici, Peptostreptococcus anaerobius, Prevotella copri, Prevotella melaninogenica, Prevotella stercorea, Propionibacterium acnes, Pseudoramibacter eubacterium, Roseburia faecis, Rothia dentocariosa, Rothia mucilaginosa, Ruminococcus bromii, Ruminococcus callidus, Ruminococcus flavefaciens, Ruminococcus lavefaciens, Ruminococcus gnavus, Ruminococcus torques, Salinibacillus aidingensis, Staphylococcus sciuri, Streptococcus anginosus, Streptococcus sobrinus, Tissierella soehngenia, Veillonella dispar, and Veillonella parvula.

In certain embodiments, one or more of the bacterial strains listed in TABLE 1 below are included in the composition.

TABLE 1

| | |
|---|---|
| Bifidobacterium adolescentis | Bacillus clausii |
| Bifidobacterium animalis | Agrococcus jenensis |
| Bifidobacterium pseudolongum | Propionibacterium acnes |
| Pseudoramibacter eubacterium | Bacteroides uniformis |
| Faecalibacterium prausnitzii | Bacteroides eggerthii |
| Dorea formicigenerans | Alistipes massiliensis |
| Blautia producta | Collinsella aerofaciens |
| Bacillus cereus | Candidatus Portiera |
| Eggerthella lenta | Coprococcus eutactus |
| Ruminococcus bromii | Collinsellas tercoris |
| Bifidobacterium longum | Prevotella melaninogenica |
| Veillonella dispar | Clostridium tyrobutyricum |
| Parabacteroides distasonis | Ruminococcus gnavus |
| Ruminococcus callidus | Anoxybacillus kestanbolensis |
| Salinibacillus aidingensis | Ruminococcus flavefaciens |
| Staphylococcus sciuri | Lactobacillus reuteri |
| Desulfosporosinus meridiei | Bifidobacterium breve |
| Lactobacillus zeae | |
| Roseburia faecis | |
| Clostridium perfringens | |
| Veillonella parvula | |

In certain embodiments, one or more of the bacterial strains listed in TABLE 2 below are included in the composition.

TABLE 2

| | |
|---|---|
| Bifidobacterium adolescentis | Eggerthella lenta |
| Bifidobacterium animalis | Veillonella dispar |
| Bifidobacterium pseudolongum | Lactobacillus zeae |
| Pseudoramibacter eubacterium | Bifidobacterium breve |
| Faecalibacterium prausnitzii | Lactobacillus reuteri |
| Dorea formicigenerans | |
| Blautia producta | |
| Bacillus cereus | |

In some embodiments, the postbiotic composition may also or as a substitute for the comprise an additional at least one non-pathogenic bacterial or fungal strains, either as a live probiotic or a postbiotic. In certain embodiments, the additional non-pathogenic bacterial organism is selected from: Bifidobacterium breve, Bifidobacterium bifidum, and Lactobacillus reuteri. In certain embodiments, an additional non-pathogenic fungal strain is selected from: Saccharomyces cerevisiae, Saccharomycetes sp HZ178, Saccharomyces bayanus, Pichia burtonii, Pichia jadinii, Pichia kudriavzevii, Pichia onychis, Pichia sp., and Picoa juniper.

In certain embodiments, the postbiotic composition comprises 1 to 30, 3 to 25, 10 to 20, or 15 billion colony forming units of any of these additional bacterial or fungal components. The composition may comprise one or more (e.g., 2, 3, 4, 5, 6, 7, 8, 9, 10, or more) different, additional non-pathogenic bacterial strains. In certain embodiments, the composition comprises in total 10 billion to 100 billion, e.g., 15 billion to 40 billion, 20 billion to 40 billion, 10 billion to 30 billion, 15 billion to 30 billion, or 20 billion to 30 billion colony forming units of the non-pathogenic bacterial strain(s).

In certain embodiments, additional exemplary fungal strains that can be included in the composition comprise any one or more of the following fungal organisms: Albatrellus syringae, Alternaria alli, Alternaria daturicola, Ambispora granatensis, Amyloathelia crassiuscula, Amylomyces rouxii, species of Ascomycota, Ascosphaera apis, Aspergillus flavus, Aspergillus fumigatus, Aspergillus oryzae, Aspergillus sp, Aspergillus terreus, Aspergillus versicolor, Bettsia alvei, Botryosphaeria mamane, Botryotinia fuckeliana, Candida arabinofermentans, Candida ernobii, Candida ethanolica, Candida glabrata, Candida humilis, Candida intermedia, Candida parapsilosis, Candida piceae, Candida quercitrusa, Candida tartarivorans, Candida temnochilae, Candida zemplinina, Candida zeylanoides, Chamonixia caespitosa, Cladonia polystomata, Cladosporium cladosporioides, Cladosporium halotolerans, Cladosporium sp JP67, Cladosporium sphaerospermum, Clavicorona taxophila, Clavispora lusitaniae, Craterellus sp, Dactylellina phymatopaga, Debaryomyces hansenii, Debaryomyces marasmus, Debaryomyces sp, Debaryomyces subglobosus, Dipodascus australiensis, Ectomycorrhizal, Emericella nidulans, Epulorhiza sp, Eremascus fertilis, Eremothecium gossypii, Eupenicillium cinnamopurpureum, Eurotium amstelodami, Eurotium cristatum, Exophiala dermatitidis, Filobasidiella neoformans, Fonsecaea monophora, Fusarium solani, Fusarium dimerum, Fusarium oxysporum, Fusarium sp, Galactomyces geotrichum, Galactomyces sp, Geomyces sp, Geotrichum cucujoidarum, Geotrichum sp, Glomus mosseae, Hanseniaspora sp, Helicobasidium longisporum, Helicostylum pulchrum, Hyphodontia flavipora, Hypochnicium cystidiatum, Inocybe sp, Kazachstania africana, Kazachstania unispora, Kluyveromyces lactis, Kluyveromyces yarrowii, Kodamaea ohmeri, Leptosphaeria biglobosa, Leptosphaerulina chartarum, Leucoagaricus sp, Lewia infectoria, Lichtheimia ramose, Macrophomina phaseolina, Monacrosporium coelobrochum, Mucor circinelloides, Mucor flavus, Mucor fuscus, Mucor sp, Myrothecium sp, Myxozyma melibiosi, Neocallimastix frontalis, Neocallimastix sp, Omphalotus nidiformis, Ophiocordyceps filiformis, Ophiocordyceps sinensis, Penicillium carneum, Penicillium chrysogenum, Penicillium concentricum, Penicillium crustosum, Penicillium digitatum, Penicillium granulatum, Penicillium griseofulvum, Penicillium griseoroseum, Penicillium polonicum, Penicillium psychrosexualis, Penicillium pulvillorum, Penicillium roqueforti, Penicillium sclerotigenum, Penicillium sp, Penicillium spinulosum, Penicillium verrucosum, Pezizomycetes sp genotype 323, Phaeophyscia exornatula, Phialocephala lagerbergii, Phlebia radiata, Phlebia sp, Phomopsis sp, Physcia stellaris, Physoderma maydis, Pichia burtonii, Pichia jadinii, Pichia kudriavzevii, Pichia onychis, Pichia sp, Picoa juniperi, Pilaira cesatii, Pilaira sp, Pirella circinans, Preussia sp, Reddellomyces donkii, Rhizomucor pusillus, Rhizopus oryzae, Rhodosporidium kratochvilovae, Rhodosporidium sp, Rhodotorula sp, Rinodina milvina, Saccharomyces bayanus, Saccharomyces bulderi, Saccharomyces cerevisiae, Saccharomyces mikatae, Saccharomyces sp, Saccharomycetes sp, Scleroderma sp, Sclerotinia sp, Scutellospora nodose, Sebacinales sp, Sporopachydermia sp, Stenocarpella maydis, Thamnidium elegans, Torulaspora delbrueckii, Trichosporon chiarellii, Tuber oligospermum, Umbelopsis isabellina, Wallemia sebi, Wallemia sp, and Zygoascus meyerae.

In certain embodiments, one or more of the fungal strains listed in TABLE 3 below may be included in the composition of the present invention.

TABLE 3

| | |
|---|---|
| Saccharomyces sp HZ178 | Pilaira sp Pi 3 |
| Mucor fuscus | Fungal endophyte sp M24 3281 |
| Saccharomyces bayanus | Candida temnochilae |
| Glomus mosseae | Cladosporium cladosporioides |
| Myxozyma cf melibiosi UWO | Galactomyces sp 3S 28C |
| Phaeophyscia exornatula | Exophiala dermatitidis |
| Sporopachydermia sp 91 1101 | Rhizomucor pusillus |
| Pichia kudriavzevii | Fungal sp K4 |
| Saccharomyces sp AS 23317 | Rhizopus oryzae |
| Galactomyces sp SDCF17 | Aspergillus fumigatus |
| Saccharomyces cerevisiae | Debaryomyces hansenii var hansenii |
| Aspergillus oryzae | Dipodascus australiensis |
| Candida arabinofermentans | Saccharomyces mikatae |

In certain embodiments, the composition may comprise a non-pathogenic fungal strain in addition to Saccharomyces boulardii, as mentioned above, selected from Saccharomyces cerevisiae, Saccharomycetes sp HZ178, Saccharomyces bayanus, strains belonging to the Pichia genus (for example, including but limited to Pichia burtonii, Pichia jadinii, Pichia kudriavzevii, Pichia onychis, and Pichia juniper).

In addition, to the at least two of Lactobacillus acidophilus, Lactobacillus rhamnosus, and Saccharomyces boulardii dead, whole-cell postbiotics, the composition may additionally comprise one or more fungal strains listed in TABLE 3. In certain embodiments, the composition of the invention comprises one or more fungal strains listed in TABLE 3 and one or more of the bacterial strains listed in TABLE 1 or TABLE 2.

In certain embodiments, the composition comprises 1 billion to 10 billion, e.g., 2 billion to 8 billion, from 3 billion to 6 million colony forming units of a non-pathogenic fungal strain(s).

In addition to the above combinations, the composition may also include combinations of enzymes (bromelain, cellulase(es), lipase(es), papain, protease(es) amylase(es), galactosidase) in the 10-250 mg range, such as, for example, 25 to 200 mg, or 50 to 150 mg. Bacopa monnieri or Bacopa monnieri extracts can be present in a 10-500 mg range, such as 25 to 300 mg, or 100 to 200 mg. In addition, Lions Mane mushroom or Lion's Mane mushroom extracts can be present in a 10-500 mg range, such as 25 to 300 mg, or 100 to 200 mg.

Other additives to any of the above combinations can be present in a total amount of 1 to 15000 mg, such as 100 mg to 10000, or 1500 to 5000 mg. Total fiber content can be present in a range of 500 mg to 5,000 mg), such as, for example, 1000 mg to 4000 mg, or 2000 mg to 3000 mg. Golden Kiwi Fiber can be present in an amount of 500 mg to 5,000 mg, such as, for example, 1000 mg to 4000 mg, or 2000 mg to 3000 mg.

In certain embodiments, the composition can be naturally sweetened, for example, with monk fruit, and preferably does not contain an allergen, artificial ingredient, or sweetener, and/or can be stored at room or ambient temperature, without refrigeration. The composition can be an engineered formulation that combines fruits, vegetables and herbal extracts with postbiotics, prebiotics and enzymes. Depending upon the circumstances, an organic super green composition can further comprise organic plant extract (e.g., Spirulina, barley grass, alfalfa leaf, wheat grass, Chlorella, dulse, spinach leaf, broccoli (whole plant), parsley leaf, kale leaf, Echinacea angustifolia root, licorice root, milk thistle seed, Siberian Eleuthero root, beet root, rose hips, acai (fruit), green tea leaf, raspberry leaf, blueberry (fruit), goji berry, bilberry (fruit), ashwagandha root, Rhodiola root, reishi mushroom, maca root, bee pollen, nettle leaf, Ginkgo biloba (4:1 leaf extract), royal jelly (3× concentrate), grape seed (2:1 extract), ginger, plant-based protein (e.g., pea protein), and fiber (e.g., sunflower lecithin, apple (fruit), brown rice bran, inulin). A nutritional ingredient may be selected from one or more of: a prebiotic (e.g., inulin), collagen peptides, deglycyrrhizinated licorice (DGL), marshmallow root, vitamin A (e.g., retinyl form), vitamin C, and vitamin D3.

In certain embodiment, the composition comprises additives such as calcium carbonate, xylitol, cetyl alcohol, citric acid, natural flavor, monk fruit. In certain embodiments, the composition comprises additives such as cascara sagrada bark, Psyllium husk, Senna leaf, Flaxseed, aloe vera leaf, licorice root, medium chain triglyceride (MCT) oil. In certain embodiments, the composition comprises additives such as a dietary fiber, e.g., inulin (fructooligosaccharides FOS) and apple pectin. In certain embodiments, the composition comprises additives such as a blend of Spirulina, barley grass, alfalfa leaf, wheat grass, Chlorella, dulse, spinach leaf, broccoli, parsley leaf, kale leaf, Echinacea angustifolia root, licorice root, milk thistle seed, Siberian eleuthero root, beet root, rose hips, acai (fruit), green tea leaf, raspberry leaf, blueberry (fruit), goji berry, bilberry (fruit), ashwagandha root, Rhodiola root, reishi mushroom, maca root, bee pollen, nettle leaf, Ginkgo biloba (leaf extract), royal jelly (3× concentrate), grape seed. In certain embodiments, the composition further comprises a vitamin, such as, vitamin C.

In certain embodiments, the composition comprises 100 mg to 2,000 mg of a prebiotic, such as inulin (e.g., 200 mg to 1500 mg, or 300 to 1000 mg). In certain embodiments, the composition comprises 1,000 mg to 20,000 mg of collagen peptides (e.g., 1,000 mg to 15,000 mg, or 2,000 to 10,000 mg). In certain embodiments, the composition comprises 100 mg to 1,000 mg of DGL or marshmallow root (e.g., 150 mg to 700 mg, or 200 to 500 mg).

In certain embodiments, the composition comprises 1,000 IU to 10,000 IU of vitamin A (e.g., 1,500 to 7,000, or 3,000 to 6,000 IU). In certain embodiments, the composition comprises 500 IU to 1,500 IU of vitamin D3 (e.g., 700 to 1,250 IU, or 850 to 1,100 IU). In certain embodiments, the composition comprises 100 mg to 1,000 mg of vitamin C (e.g., 200 mg to 800 mg, or 250 to 600 mg).

In certain embodiments, the composition comprises 1,000 mg to 20,000 mg of a plant-based protein, such as pea protein (e.g., 1,200 mg to 15,000 mg, or 2,000 mg to 5,000 mg). In certain embodiments, the composition comprises about 10 mg to about 200 mg of grape seed extract (e.g., 20 mg to 150 mg, 45 mg to 100 mg). In certain embodiments, the composition comprises 100 mg to 1,000 mg of ginger (e.g., 125 mg to 750 mg, or 250 mg to 550 mg).

In certain embodiments, a composition of the composition may further comprises one or more of the following ingredients, including Spirulina, Chlorella, dulse, barley grass, alfafa juice concentrate, wheat grass, spinach leaf, kale leaf, beet root, rose hips, acai fruit, raspberry fruit, blueberry fruit, goji berry, bilberry fruit extract, green tea leaf extract, Ginkgo biloba, Echinacea purpurea root, licorice root extract, milk thistle extract, stinging nettle root, royal jelly, grape seed extract, bee pollen, maca root, reishi mushroom, Rhodiola root, ashwagandha root, and/or fiber (e.g., sunflower lecithin, apple fiber, brown rice bran). In certain embodiments, a composition of the present invention can be flavored using Stevia or monk fruit, or another natural flavor (e.g., natural blueberry flavor).

The compositions or dosage forms described herein, can be administered or consumed individually or in combination. For example, a composition that includes the Lactobacillus rhamnosus, Bifidobacterium longum, Green tea, Cranberry extract, Nichi glucan, glucans produced from Aureobasidium pullulans, polysaccharides produced from Aureobasidium pullulans, or β-1,3-1,6-glucans, and BIOHM FX may be administered or consumed individually as such, or may be co-administered or concurrently consumed with other nutrients or supplements.

In another aspect, the composition or method of treatment can comprise a nutrient in combination with the postbiotic composition. The combination does not require that the nutrient and the postbiotic composition be present in a single composition. In certain embodiments, the composition comprising a nutrient comprises both the nutrient and the postbiotic composition.

In certain embodiments, the consumption by, or administration to the subject of the composition comprising a nutrient is contemporaneous with the consumption or administration of the postbiotic composition. In fact, the postbiotic form allows for easier incorporation of the composition into foods and drinks, including baked and/or cooked foods.

In certain embodiments, a nutrient is included in the composition. In an embodiment, the nutrient is a protein. The protein may be selected from whey protein, soy protein, milk protein, egg protein, animal muscle protein, fish protein, brown rice protein, pea protein, hemp protein, cranberry protein, and artichoke protein. In certain embodiments, the protein is casein. In certain embodiments, the protein is a protein hydrolysate. In certain embodiments, the protein is comprised in a natural food source. In certain embodiments, the protein is an isolated and/or purified protein.

In another aspect, the nutrient may comprise a beverage product comprising a postbiotic composition comprising the postbiotic and enzyme components discussed above. The beverage can be a protein- or fiber-rich beverage, e.g., comprising at least 3%, 4%, 5%, 6%, 7%, 8%, 9%, or 10% of protein or prebiotic fiber. The postbiotic composition can include one or more of the compositions or dosage forms described herein.

The postbiotic composition can be incorporated into cooked, for example, baked, food products. Exemplary baked food products may include but are not limited to: breads, buns, bagels, cakes, cookies, crackers, pancakes, muffins, biscuits, pies, brownies, casseroles, puddings, or tarts.

Given that the cells of the postbiotic are already dead, the baking process for producing a baked product does not materially compromise the efficacy of the postbioitic composition contained therein. In an embodiment, the microbial strains (non-pathogenic fungal and non-pathogenic bacterial strains) and/or an enzyme included in the postbiotic composition contained in the baked food product survive baking temperatures up to 450° F. (e.g., 200, 250, 300, 350, 400, or 450° F.) for up to 300 minutes (e.g., 20 to 90, 30 to 240, or 60 to 180 minutes). As a result, the completed baked food products contain postbiotic compositions with effective amounts (non-pathogenic fungal and bacterial strains) and optionally an enzyme for improving immune health.

It is contemplated that the cooked food product is protein or fiber-rich, e.g., comprising at least 25%, 50%, about 2-fold, about 3-fold, about 4-fold, or about 5-fold more protein per unit weight than average baked food products of the kind, or at least 20%, 25%, 30%, 35%, 40%, 45% or 50% of fiber for consumption by subjects with biofilm as well as healthy subjects. In embodiments, fiber may be selected as disclosed elsewhere in this document. In certain embodiments, the cooked food product comprises a protein selected from whey protein, soy protein, milk protein, egg protein, animal muscle protein, fish protein, brown rice protein, pea protein, hemp protein, cranberry protein, and artichoke protein. In certain embodiments, the cooked food product comprises a milk protein, optionally wherein the milk protein is casein. In certain embodiments, the protein is a natural protein. In certain embodiments, the protein is a protein hydrolysate (e.g., a partial hydrolysate of a natural protein). The protein can be comprised in a natural food source (e.g., in milk or dairy product, soy, egg, animal muscle, fish, brown rice, or pea) used in preparation of the cooked food product. Alternatively, the protein can be isolated and/or purified from a natural food source and added to the cooked food product.

The cooked food product can be produced by cooking one or more food ingredients with a postbiotic composition. Accordingly, the invention also provides food preparation kits comprising one or more food ingredients and the postbiotic composition disclosed herein. The food ingredients and the postbiotic composition can be packaged separately or in a mixed composition. If packaged separately, the food preparation kit can further comprise an instruction sheet, in a printed or electronic form, to mix the postbiotic composition with the food ingredients prior to or after cooking.

The postbiotic composition can be included in a beverage product that contain any of the compositions described herein. Exemplary beverage products may include but are not limited to: protein shakes, protein smoothies, nutritional drinks, and sports drinks.

It is contemplated that the composition can be added to a protein- or fiber-rich beverage, e.g., comprising at least 3%, 4%, 5%, 6%, 7%, 8%, 9%, or 10% of protein or at least 20%, 25%, 30%, 35%, 40%, 45% or 50% of fiber, for consumption by subjects with biofilm as well as healthy subjects. In embodiments, the fiber may be selected as mentioned elsewhere in this document. In embodiments, the beverage product comprises a protein selected from whey protein, soy protein, milk protein, egg protein, animal muscle protein, fish protein, brown rice protein, pea protein, hemp protein, cranberry protein, and artichoke protein. In certain embodiments, the beverage product comprises a milk protein, optionally wherein the milk protein is casein. In certain embodiments, the protein is a natural protein. In certain embodiments, the protein is a protein hydrolysate (e.g., a partial hydrolysate of a natural protein). The protein can be comprised in a natural food source (e.g., in milk or dairy product, soy, egg, animal muscle, fish, brown rice, or pea) used in preparation of the beverage product. Alternatively, the protein can be isolated and/or purified from a natural food source and added to the beverage product. In certain embodiments, the beverage product further comprises a nutrient selected from the group consisting of a carbohydrate (e.g., glucose, fructose, sucrose, maltose, or maltodextrin), an electrolyte (e.g., sodium, chloride, potassium, calcium, or magnesium), a vitamin (e.g., vitamin B3, vitamin B6, vitamin B12, vitamin C, vitamin D, vitamin E), an antioxidant (e.g., pomegranate juice, berry juice, tea, caffeine), citrulline, and carnitine.

The beverage product can be produced by mixing one or more beverage ingredients with the postbiotic composition. Accordingly, beverage preparation kits may comprise one or more beverage ingredients and the postbiotic composition disclosed herein. The beverage ingredients and the postbiotic composition can be packaged separately or in a mixed composition. If packaged separately, the beverage preparation kit can further comprise an instruction sheet, in a printed or electronic form, to mix the postbiotic composition with the beverage ingredients.

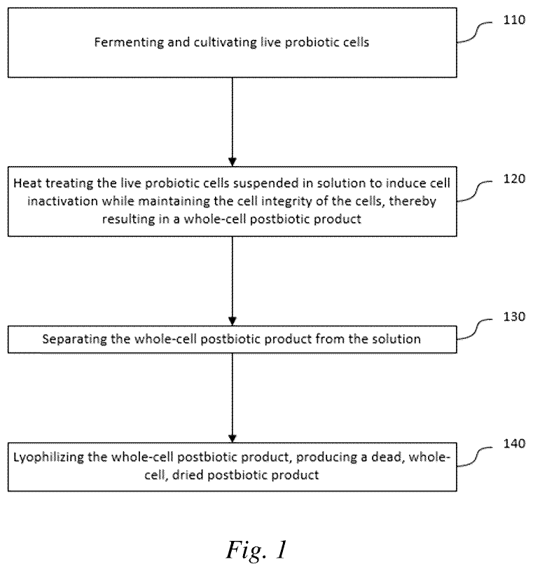

FIG. 1 is a flowchart of an example method for of making a postbiotic product. The process of making the postbiotic product involves the fermentation of live probiotic cells, followed by their heat-induced inactivation in solution, subsequent separation through centrifugation to form a pellet, and concluding with freeze-drying to produce a final powdered product.

The process commences at step 110 with the fermentation and cultivation of live probiotic cells within a suitable growth medium. These cells proliferate under controlled conditions to attain the desired biomass concentration. The live probiotic cells are selected from two or more of the following components: Lactobacillus acidophilus, Lactobacillus rhamnosus, and Saccharomyces boulardii. In an embodiment, the live cells can be sourced from various sources and are then each cultured separately in a small scale fermenter. For each strain, a culture sample is then used to inoculate a corresponding large scale production fermenter. Suitable growth mediums include broths with milk, yogurt, tapioca, or meat-derived ingredients, vegetable based broths may also be used such as those containing soy peptone, yeast extract and glucose monohydrate. The different species of cells can be fermented together, or fermented separately and then mixed after this step or later in the process.

Following the fermentation phase, at step 120, the live probiotic cells are subjected to a controlled heat treatment while suspended in solution. This heat treatment serves to induce cell inactivation while maintaining the whole-cell integrity of cellular structures. In some embodiments, the one or more organisms are rendered non-replicating by high-temperature treatment. In some embodiments, the high temperature treatment is a high temperature/short time (HTST) treatment or an ultra-high temperature (UHT) treatment, as those terms are known in the art. The different species of cells can be heat treated together or separately and then mixed after this step or later in the process. The organisms are rendered dead by this process and are now referred to as postbiotics.

At step 130, the heat-treated postbiotic cell suspension is then subjected to separation. centrifugation. This can involve washing, centrifugation, and/or other separation processes. In an embodiment, the cells are washed by pumping fermentation broth into a sterile saline broth tank. Centrifugation can be used with or without washing, wherein centrifugal forces segregate the cell biomass from the surrounding medium. Properly done, with sufficient rpms and with sufficient time, this results in the formation of a distinct cell pellet at the bottom of the centrifugation vessel. Heat treatment, e.g., in an industrial oven or dryer, could also continue to evaporate the solution to aid in or complete the separation.

At step 140, the obtained whole-cell postbiotic pellet is subsequently subjected to lyophilization. This involves a multi-step process where the cell pellet is first frozen, followed by the sublimation of ice crystals under reduced pressure. This sublimation process effectively removes moisture from the cells, preserving their structural and functional characteristics.

The culmination of the process yields a powdered product consisting of heat-inactivated postbiotic whole cells. This freeze-dried powder possesses an extended shelf life due to the removal of moisture, thereby rendering it stable at ambient conditions. If desired, other ingredients specific to the formulation (e.g., dried amylase, other pro- or postbiotics, optionally a dried BIOHM FX component) can be added. If desired, the blended composition can then be encapsulated, which can then be packaged into an appropriately labeled container. The powder can be encapsulated in a dosage form, such as a pill or capsule. The powder can be mixed in food products, such as, for example, dry mixes for foods or drinks. Other food products are described below.

In an embodiment, the postbiotic composition can be used to treat or provide to a subject an immunomodulatory, immune regulatory and/or immune boosting effect, the method comprising consumption by, or administration to, the subject the postbiotic composition. The Examples below demonstrated that this effect can be expected in vivo, through in vitro real time qPCR measuring the amount of mRNA for pro-inflammatory cytokine genes (at least 2 fold increase in expression of IL6 and TNF genes over a negative control). In an embodiment the postbiotic composition can produced a 2 to 10 fold increase in expression of IL6 and TNF genes over a negative control, such as 3 to 8 fold, or 3.5 to 6 fold. Further confirmation was obtained through a Luminex 45-plex cytokine, chemokine, and growth factor panel analyzed after incubating the postbiotic combinations described above with human monocyte (CD14+) cells for 24 hours. The postbiotic compound was also found capable of stimulating cytokine gene expression using an in vitro enterocyte model, including changes in Tumor Necrosis Factor alpha (TNFa).

In an embodiment, the postbiotic composition can be used to treat a disorder of the gut barrier or improve a gat barrier. The gut barrier is a mucosal lining in the intestines of a subject. The subject can have or be susceptible to, for example, a disorder such as, irritable bowel syndrome, inflammatory bowel disease, intestinal allergic syndrome or celiac sprue. The subject can be at risk for developing gut barrier dysfunction or an illness associated with gut barrier dysfunction due to, for example, a diet deficient in nutrients, exposure to a toxin, an parasitic or bacterial infection, dysbiosis, or long-term antibiotic usage. In an embodiment, a method of improving a gut barrier comprises consumption by, or administration to, a subject a composition comprising postbiotics comprising two or more of the following components: Lactobacillus rhamnosus, and Saccharomyces boulardii; wherein the postbiotics are dead whole-cell postbiotics. In an embodiment, the improving of the gut barrier comprises reducing TNFa gene expression by the composition as compared to stimulation with lipopolysaccharide from E. coli

In an embodiment, a method includes consumption by, or administration to, a subject a composition comprising postbiotics comprising the following components: Lactobacillus rhamnosus and Saccharomyces boulardii; wherein the postbiotics are dead whole-cell postbiotics. Improving of the gut barrier may be determined by a reduction in the TNFa gene expression by the composition as compared to stimulation with lipopolysaccharide from E. coli. This can be determined according to the method disclosed in Ruder B, Atreya R, Becker C. “Tumour Necrosis Factor Alpha in Intestinal Homeostasis and Gut Related Diseases,” Int J Mol Sci. 2019 Apr. 16; 20(8):1887. doi: 10.3390/ijms20081887. PMID: 30995806; PMCID: PMC6515381. “Increased TNFα levels can trigger epithelial cell death in the intestine, which potentially leads to barrier defects and invasion of harmful pathogens. Altogether this might trigger or perpetuate the development of intestinal inflammation.” Id.

Compositions or dosage forms are provided herein that can improve immune response. In certain embodiments, the composition is formulated as a powdered blend of the components described above that optionally is coated with a functional coating (e.g., controlled release coating) or a non-functional coating (e.g., aesthetic coating). Controlled release coatings can facilitate the continuous release, gradual release, prolonged release, and/or programmed release (e.g., pH-dependent release) of the microorganisms in the compositions or dosage forms disclosed herein. In certain embodiments, controlled release coating is hydroxypropyl methylcellulose (HPMC).

While typically the composition is designed for human subjects, in certain situations, the subject may be a non-human mammal (e.g., human, a companion animal (e.g., dog, cat, or rabbit), or a livestock animal (for example, cow, sheep, pig, goat, horse, donkey, and mule, buffalo, oxen, or camel)). In certain embodiments, the subject is a healthy subject.

In certain embodiments, 30-50 of such capsules (e.g., 35-45 capsules) can be consumed over a period of 2-8 weeks (e.g., 3-6 weeks). In certain embodiments, the product can be consumed by a subject for up to one month (e.g., 30 days), or repeated cycles of one month. In each cycle, a single serving size (e.g., 1 scoop) may be consumed per day. For example, one capsule can be consumed per day, with or without the simultaneous consumption of food. In an embodiment, the postbiotic composition does not contain an allergen, artificial ingredient, or sweetener, and/or can be stored at room or ambient temperature, without refrigeration.

In certain embodiments, the composition may be consumed orally by the subject. In certain embodiments, the composition is consumed as a powder or product containing the powder. In other embodiments, the composition can be in the form of an oral dosage form, for example, where the composition in included, for example, within a capsule, cachet, pill, tablet, lozenge, powder, granule, or as a solution or a suspension in an aqueous or non-aqueous liquid, or as an oil-in-water or water-in-oil liquid emulsion, or as an elixir or syrup, or as pastilles, each containing the requisite number of colony forming units of the non-pathogenic bacteria and non-pathogenic fungus, and optionally the appropriate amount of enzyme.

In certain embodiments, the composition is disposed in a capsule or in a tablet. In certain embodiments, the capsule is a vegetable cellulose capsule. In certain embodiments, the dosage form, for example, capsule or tablet, is coated with a coating, for example, a non-functional aesthetic coating or a functional coating, for example, a controlled release coating. The capsule or tablet may be formulated so as to provide slow or controlled release of the ingredients disposed therein.

The size of the dose delivered or consumed, will depend upon, among other things, the size and age of the subject, the indication or condition to be treated, and the mode of delivery of the composition. In certain embodiments, for example, oral compositions may be 1 mg to 10,000 mg or 50 mg to 10,000 mg of the active ingredients (for example, the non-pathogenic bacteria, the non-pathogenic fungus, and/or the enzyme). In certain embodiments, the composition may be 1 mg to 9,000 mg, 1 mg to 8,000 mg, 1 mg to 7,000 mg, 1 mg to 6,000 mg, 1 mg to 5,000 mg, 1 mg to 4,000 mg, 1 mg to 3,000 mg, 1 mg to 2,000 mg, 1 mg to 1,000 mg, 1 mg to 500 mg, 10 mg to 1000 mg, 20 mg to 1000 mg, 30 mg to 1000 mg, 40 mg to 1000 mg, 50 mg to 1000 mg, 100 mg to 1000 mg, 200 mg to 1000 mg, 300 mg to 1000 mg, 400 mg to 1000 mg, 500 mg to 1000 mg, 600 mg to 1000 mg, 700 mg to 1000 mg, 800 mg to 1000 mg, or 900 mg to 1000 mg of the active ingredients (for example, those disclosed above or in the Examples).

The compositions (either one or multiple units (for example 2, 3, 4, or 5 units, for example, capsules or tablets) may be administered once, twice or three times a day during the treatment period, for example, until the biofilm of interest has been disrupted, prevented from forming, and/or the subject's normal microbiome has been restored which could take, for example, one week, two weeks, one month, two months, or three months and one year. In certain embodiments, one capsule, such as one enteric-coated capsule, that comprises about 30 billion colony forming units of non-pathogenic fungal strain(s) and non-pathogenic bacterial strain(s) and an enzyme, is administered once, twice, or three times a day during the treatment period, for example, until the biofilm of interest has been disrupted and/or the subject's normal microbiome has been restored which could take, for example, one week, two weeks, one month, two months, or three months and one year.

In certain embodiments, the composition may be formulated for topical delivery, for example, as a liquid, emulsion, suspension, ointment, cream, gel, lotion, or powder. In certain embodiments, upon application to a skin of a subject, the composition may form a patch. Depending on the concentration of non-pathogenic organisms and the particular delivery device, the composition may be formulated to deliver from 1.0 mL/5 cm² to 1.0 mL/50 cm², or from 1.0 mL/5 cm² to 50 mL/50 cm², or from 1.0 mL/5 cm² to 100 mL/50 cm².

The topical dosage form may also include a pharmaceutically acceptable carrier. Suitable carriers that may be useful in topical formulations include, but are not limited to, solubilizers such as C₂-C₈, straight and branched chain alcohols, diols and triols, moisturizers and humectants such as glycerin, amino acids and amino acid derivatives, polyaminoacids and derivatives, pyrrolidone carboxylic acids and its salts and derivatives, surfactants such as sodium lauryl sulfate, sorbitan monolaurate, emulsifiers such as cetyl alcohol, stearyl alcohol, thickeners such as methyl cellulose, ethyl cellulose, hydroxymethylcellulose, hydroxypropylcellulose, polyvinylpyrollidone, polyvinyl alcohol and acrylic polymers.

In certain embodiments, the composition may be formulated for nasal or oral administration in the form of a nasal spray and inhalation solution, and suspension, that is administered for example, by inhalation through the nose. The dose can be delivered to the nasal cavity via a spray pump or as nasal drops for local and/or systemic effects.

In certain embodiments, the composition may be an inhalation solution and/or suspension for delivery to the upper and lower respiratory tract (e.g. oropharynx, lungs) by oral inhalation for local and/or systemic effects and can be used with a specified nebulizer.

Other compositions, components, concentrations, and dosages that can be combined with the teachings herein are disclosed in U.S. 2019/0381119 and U.S. 2021/0030818, incorporated herein by reference.

The following definitions are included for the purpose of understanding the present subject matter and for constructing the appended patent claims.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

Further, it should be understood that elements and/or features of a composition or a method described herein can be combined in a variety of ways without departing from the spirit and scope of the present disclosure, whether explicit or implicit herein. For example, where reference is made to a particular composition, that composition can be used in the various embodiments of such compositions of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present invention also includes the specific quantitative value itself, unless specifically stated otherwise. As used herein, the term “about” refers to a ±10% variation from the nominal value unless otherwise indicated or inferred.

As a general matter, compositions specifying a percentage are by weight unless otherwise specified.

As used herein, the term “nutritional ingredient” refers to one or more ingredient(s) added to a composition of the present disclosure, which can be a natural ingredient having no known adverse physiological effect in a human, or known to be beneficial or necessary for optimal physiology and health of a human.

As used herein, the terms “treat,” “treating,” or “treatment,” and other grammatical equivalents as used herein, include alleviating, abating, ameliorating, or preventing a disease, condition or symptoms, preventing additional symptoms, ameliorating or preventing the underlying causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition, and are intended to include prophylaxis.

As used herein, the terms, “subject,” “patient,” “subject in need thereof,” and “patient in need thereof” are used interchangeably herein, and refer to a living organism, including animals and humans, suffering from or prone to a disease or condition that can be treated by the methods and compositions provided herein. The subject can be a human or non-human animal.

The foregoing examples are presented herein for illustrative purposes only, and should not be construed as limiting the invention in any way.

### EXAMPLES

The following Examples are merely illustrative and are not intended to limit the scope or content of the invention in any way.

### Example 1

Various postbiotic compositions were made according to the process disclosed in FIG. 1 and explained above. The compositions were either washed or centrifuged only as explained in step 130.

Primary human CD14+ monocytes were exposed in wells to each postbiotic composition individually at a concentration of 100 μg/mL. Monocytes are agranular leukocytes of the myeloid lineage and are present in circulating white blood cells. Such monocytes are characterized by the expression of the CD14 cell surface receptor and play a role in the immune response to foreign pathogens. Probiotic organisms, e.g., probiotic cell walls can stimulate the adaptive immune system by activating the cytokine network. Probiotics can be regulators of the immune system and shape both innate and adaptive immune response.

A negative control example (NEG) was provided with a set of wells, containing CD14+ monocytes not exposed to any stimuli. A positive control example (LPS) contained 10 ng/mL of liposaccharides (LPS) from enterohemorrhagic E. coli. LPS is a known positive control for inducing cytokine response. A control example with just tapioca starch was also included.

Examples of the whole-cell, non-viable postbiotics were tested at a concentration of 100 μg/mg in phosphate buffered saline. (They were diluted 1:100 in each well of a bank of wells for a final concentration of 100 μg/mg and exposed to the primary human CD14+ monocytes for 24 hours.) Each sample well contained 200,000 CD14+ cells. CD14+ cells were incubated in RPMI 1640 with Glutamate, 10% Fetal Bovine Serum, and 1% Penicillin/Streptomycin.

Certain examples were washed, others were not washed, and some were washed in house. The inhouse wash was a small scale saline wash to determine if the unwashed or washed cells were performing better while scale up washed production samples were being made. Whole cell, non-viable, postbiotic compositions of LA (L. acidophilous) and LR (L. rhamnoses) were tested in 1:1, 1:2, and 2:1 ratios. LA and LR were also tested alone and not in combination.

After 24 hours of exposure, RNA was harvested from each of the wells. Real time qPCR was performed to measure the amount of mRNA for four genes: GaPDH (a housekeeping gene used to normalize RNA between samples), TNF-alpha (a pro-inflammatory cytokine), IL10 (an anti-inflammatory cytokine; and IL6 (a pro-inflammatory cytokine). The Ct value for each of these genes was determined using a singleplex assay format. The fold change was then calculated in gene expression for the cytokines using the delta delta Ct method.

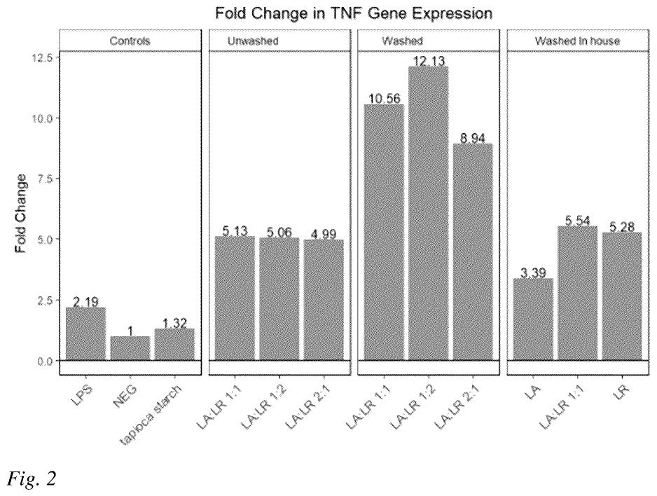

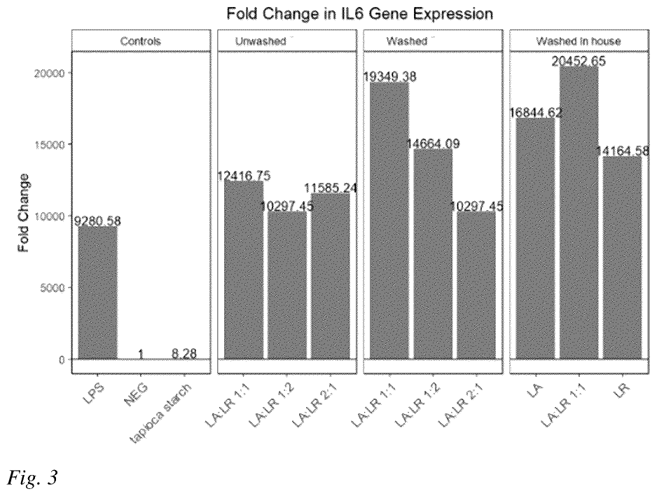

FIGS. 2 and 3 are graphs of control examples and unwashed, washed, and washed-in-house examples for fold change in TNF and IL6 gene expression. For TNF, while all examples showed at least 3.39 fold change compared to at most 2.19 fold change in the control examples, the washed examples were the best performers. For IL6 all examples showed at least 10297.45 fold change over the negative example, and all were improved by over 1000 fold compared to the LPS control example.

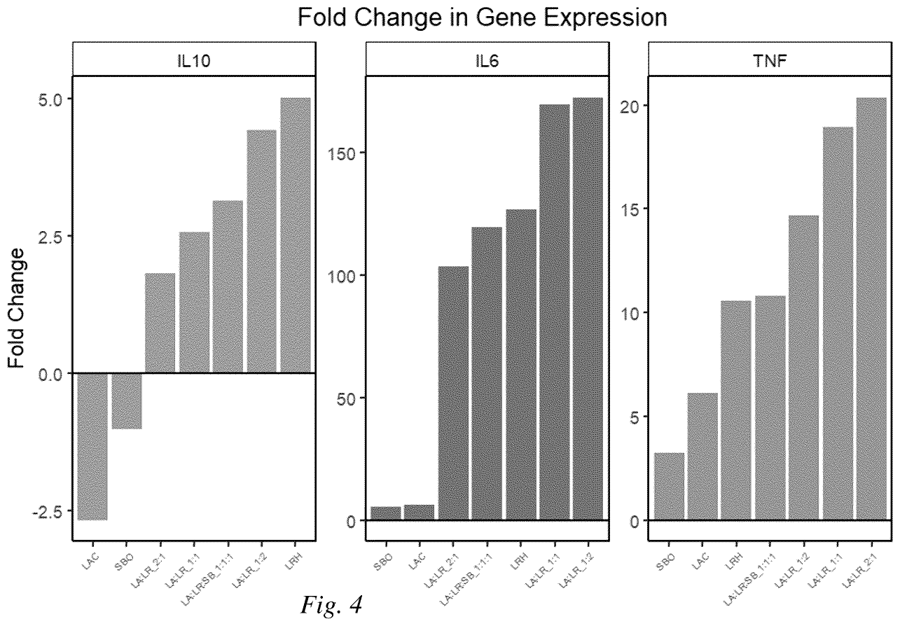

FIG. 4 is a graph showing other combinations of postbiotic components and the fold change in gene expression. In FIG. 4, LAC is Lactobacillus acidophilus, LRH is Lactobacillus rhamnosus, SBO is S. boulardii. For combinations, LA is Lactobacillus acidophilus, LR—Lactobacillus rhamnosus, SB—Saccharomyces boulardii. Notably, S. boulardii is included in this data and IL10 data is included. Also, of note, alone, SB and LAC had little to negative fold change, especially for both IL10 and IL6. However, their use in combination results in a synergistic benefit in fold change especially in IL10 and IL6 counts.

### Example 2

Additional data was gathered through a Luminex 45-plex cytokine, chemokine, and growth factor panel analyzed after incubating the postbiotic combinations described above with human monocyte (CD14+) cells for 24 hours. The Luminex assay is used for quantifying the amount of various soluble growth factors, chemokines, and cytokines expressed by the target cell of interest, in this case human monocytes.

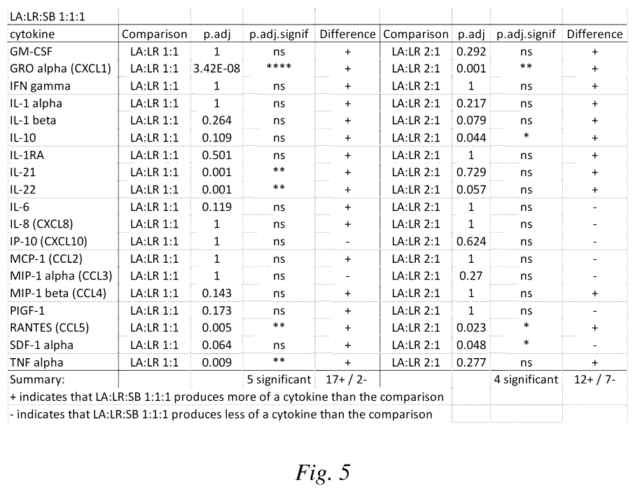

FIGS. 5-7 present a data summary comparing the cytokine profiles of the monocytes treated with either single species or multiple-species postbiotic compositions. The cytokine profiles of the multi-species postbiotics were compared to each other, as well as to the single species postbiotics. Targets of interest were selected to be molecules that have contextual sense for a monocyte product, as well as those that exceed a minimal threshold for response. These target cytokines are listed in the left hand column of the graphs.

FIG. 5 shows data for postbiotic compositions comprising LA and LR in 1:1 and 2:1 ratios and compares these to an LA:LR:SB 1:1:1 composition. Overall, it can be seen that the LA:LR:SB composition promotes a greater cytokine response than the LA:LR compositions. The concentrations of cells (1:1:1 and 1:1) and test conditions of these tested compositions were equal.

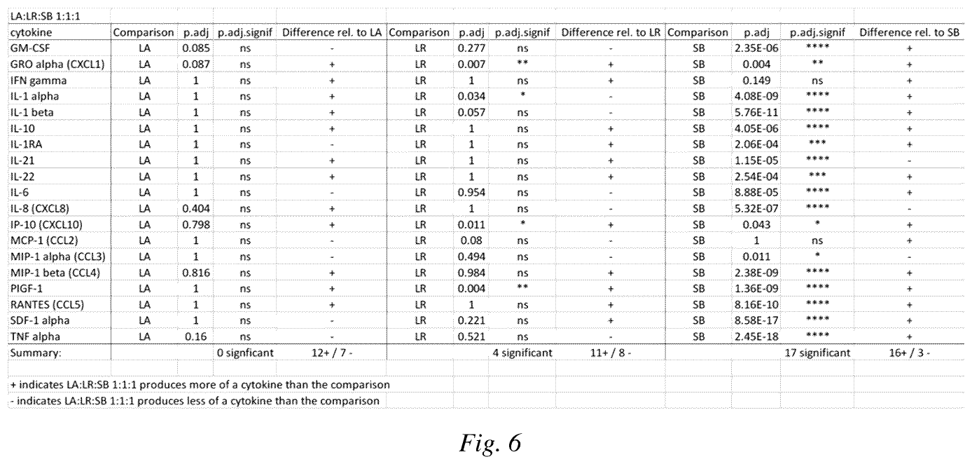

FIG. 6 shows data for postbiotic compositions comprising LA:LR:SB in a 1:1:1 ratios and compares these to an LA, LR, and SB separately. Overall, it can be seen that the LA:LR:SB composition promotes a greater cytokine response than the separate compositions. The concentrations and test conditions of these tested compositions were equal (1:1:1).

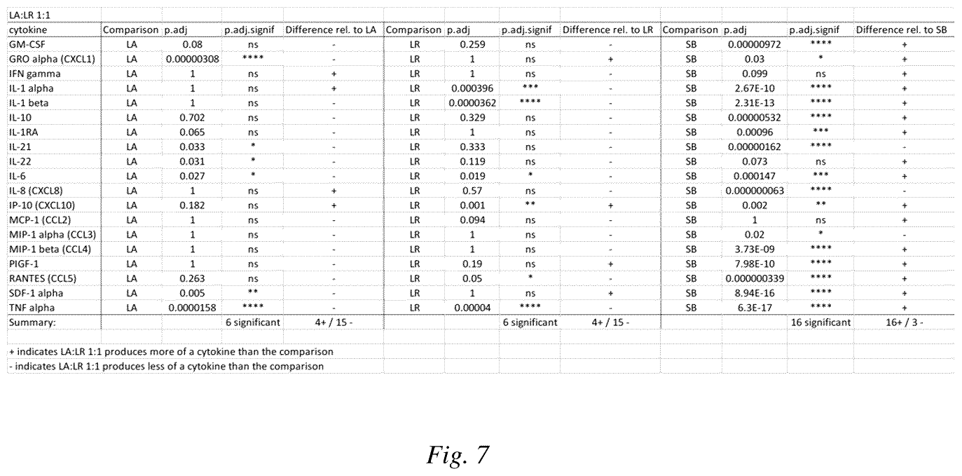

FIG. 7 shows data for postbiotic compositions comprising LA and LR in 1:1 ratios and compares these to an LA, LR, and SB separately. Overall, it can be seen that the LA:LR composition promotes a greater cytokine response than the LA, LR, and SB compositions separately. The concentrations and test conditions of these tested compositions were equal.

Overall, FIGS. 2-7 illustrate a synergistic improvement in the combination of LR and LA, and LA, LR, and SB over what would be expected from the single compounds themselves in terms of cytokine immune response in a human monocyte.

### Example 3

The ability of a postbiotic compound as disclosed herein comprised of heat-killed microbial cells and cell wall components to stimulate cytokine gene expression in an in vitro enterocyte model was also investigated. It was found that the postbiotic compound was capable of stimulating cytokine gene expression using this model, including changes in Tumor Necrosis Factor alpha (TNFa). TNFa is known to induce a disruption of barrier integrity of the intestinal epithelium in vivo, therefore, a postbiotic that is capable of reducing TNFa gene expression could potentially have barrier-promoting abilities. We observed that both Saccharomyces cerevisiae var. boulardii and Lacticaseibacillus rhamnosus both reduced TNFa gene expression as compared to stimulation with lipopolysaccharide from E. coli. This effect was dose-dependent, suggesting that cell wall components from both of these organisms may also be capable of preventing a loss of barrier integrity in response to a proinflammatory stimulus.

The entire disclosure of each of the patent and scientific documents referred to herein is incorporated by reference.

What has been described above includes examples of one or more embodiments. It is, of course, not possible to describe every conceivable modification and alteration of the above devices or methodologies for purposes of describing the aforementioned aspects, but one of ordinary skill in the art can recognize that many further modifications and permutations of various aspects are possible. Accordingly, the described aspects are intended to embrace all such alterations, modifications, and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim. The term “consisting essentially” as used herein means the specified materials or steps and those that do not materially affect the basic and novel characteristics of the material or method. Unless the context indicates otherwise, all percentages and averages are by weight. If not specified above, the properties mentioned herein may be determined by applicable ASTM standards, or if an ASTM standard does not exist for the property, the most commonly used standard known by those of skill in the art may be used. The articles “a,” “an,” and “the,” should be interpreted to mean “one or more” unless the context indicates the contrary.

What is claimed is:

1. A composition comprising:
postbiotics comprising two or more of the following components: Lactobacillus acidophilus, Lactobacillus rhamnosus, and Saccharomyces boulardii; wherein the postbiotics are dead, whole-cell postbiotics.

2. The composition of claim 1, wherein the postbiotics comprise Lactobacillus acidophilus, Lactobacillus rhamnosus, and Saccharomyces boulardii, wherein the postbiotics are dead, whole-cell postbiotics.

3. The composition of claim 1, wherein postbiotics comprise Lactobacillus acidophilus and Lactobacillus rhamnosus, wherein the postbiotics are dead, whole-cell postbiotics.

4. The composition of claim 1, wherein the postbiotics are heat killed.

5. The composition of claim 1, further comprising a prebiotic fiber.

6. The composition of claim 5, wherein the prebiotic fiber is selected from the group consisting of one or more of: arabinoxylan, beta-glucan, carboxymethylcellulose, cellulose, chicory root fiber, cottonseed fiber, edible bean powder, fructo-oligosaccharides, galacto-oligosaccharides, hydroxypropyl-methylcellulose, inulin (artichoke), inulin (chicory), methylcellulose, modified resistant starch, oligofructose, pea fiber, pectin, polydextrose, polyfructans, Psyllium, resistant starch, resistant dextrin, resistant maltodextrin, rice bran, short chain fructooligosaccharides, soluble corn fiber, soluble dextrin, soy fiber, wheat bran, xanthan gum, golden kiwi fiber, xylo-oligosaccharides, galactomannan (guar gum) and tapioca starch.

7. The composition of claim 1, further comprising Lactobacillus acidophilus, Lactobacillus rhamnosus, Saccharomyces boulardii, Bifidobacterium breve, and alpha amylase in viable probiotic form.

8. The composition of claim 1, wherein the postbiotic composition further comprises an enzyme that disrupts extracellular matrix polysaccharides (EPSs) present in a gastrointestinal tract biofilm.

9. The composition of claim 8, wherein the enzyme is an amylase.

10. The composition of claim 1, wherein the components of the composition are present in effective amounts to improve immune response as determined by in vitro exposure to human monocytes CD14+ and at least 2 fold increase in expression of IL6 and TNF-alpha genes over a negative control.

11. The composition of claim 1, further comprising one or more additional probiotic components selected from the group consisting of: Lactobacillus casei, Lactobacillus plantarum, Lactobacillus acidophilus, Bacillus subtillis, Lactobacillus rhamnosus, Lactobacillus helveticus, Bifidobacterium longum, Bifidobacterium lactis, Bifidobacterium infantis, Bifidobacterium bifidum, Bifidobacterium adolescentis, Bifidobacterium breve, Saccaromyces boulardii, Brevebacillus laterosporus, and combinations thereof.

12. A method of providing to a subject an immunomodulatory, immune regulatory and/or immune boosting effect, the method comprising consumption by, or administration to, the subject a composition comprising postbiotics comprising two or more of the following components: Lactobacillus acidophilus, Lactobacillus rhamnosus, and Saccharomyces boulardii; wherein the postbiotics are dead whole-cell postbiotics.

13. The method of claim 12, wherein the postbiotics comprise Lactobacillus acidophilus, Lactobacillus rhamnosus, and Saccharomyces boulardii; wherein the postbiotics are dead whole-cell postbiotics.

14. The method of claim 12, wherein the postbiotics comprise Lactobacillus acidophilus and Lactobacillus rhamnosus, wherein the postbiotics are dead, whole-cell postbiotics.

15. The method of claim 12, wherein the composition further comprises administering a probiotic composition comprising Lactobacillus acidophilus, Lactobacillus rhamnosus, Saccharomyces boulardii, Bifidobacterium breve, and alpha amylase in viable probiotic form.

16. The method of claim 12, wherein the components of the composition are present in effective amounts to improve immune response as determined by in vitro exposure to human monocytes CD14+ and at least 2 fold increase in expression of IL6 and TNF-alpha genes over a negative control.

17. A method of making a postbiotic product, the steps comprising:
fermenting and cultivating live probiotic cells, the cells selected from two or more of the following components: Lactobacillus acidophilus, Lactobacillus rhamnosus, and Saccharomyces boulardii;
heat treating the live probiotic cells suspended in solution to induce cell inactivation while maintaining integrity of the cells, thereby resulting in a whole-cell postbiotic product;
separating the whole-cell postbiotic product from the solution; and
wherein the steps are done separately for each of the two or more cells selected from: Lactobacillus acidophilus, Lactobacillus rhamnosus, and Saccharomyces boulardii; and
mixing the whole-cell postbiotic products.

18. The method of claim 17, wherein the separating the whole-cell postbiotic product includes washing the product.

19. The method of claim 17, further comprising lyophilizing the whole-cell postbiotic product, producing a dead, whole-cell, dried postbiotic product.

20. The method of claim 18, wherein the separating the whole-cell postbiotic product includes centrifugating the washed product.
